(19) United States
(12) Patent Application Publication
(10) Pub. No.: US 2024/0415786 A1
(43) Pub. Date: Dec. 19, 2024

(54) COMPOSITIONS AND METHODS USING AN AUTOPHAGY INDUCER TO ENHANCE INTERMITTENT FASTING

(71) Applicant: SOCIETE DES PRODUITS NESTLE S.A., Vevey (CH)

(72) Inventors: CLAIRE REGARD (BOUTRY), Thevenon (CH); GABRIELE CIVILETTO, Lausanne (CH); PHILIPP GUT, Geneve (CH); JEROME FEIGE, Crissier (CH)

(21) Appl. No.: 18/703,966

(22) PCT Filed: Oct. 26, 2022
(86) PCT No.: PCT/EP2022/079998

§ 371 Date: Apr. 23, 2024

Related U.S. Application Data
(60) Provisional application No. 63/272,229, filed on Oct. 27, 2021.

(51) Int. Cl. A61K 31/05; A23L 33/00; A61P 39/00

(52) U.S. Cl. CPC A61K 31/05; A23L 33/30; A61P 39/00

(57) ABSTRACT

Compositions contain an autophagy inducer and are administered before, during and/or after an intermittent fasting (IF) diet, preferably a time-restricted feeding (TRF) regimen or an alternate day fasting (ADF) regimen. For example, enhanced intermittent fasting provided by the compositions and methods disclosed herein can include improvement of at least one of longevity, cardiometabolic health, body composition (e.g., fat mass reduction), cellular ageing (e.g., reduction in inflammation markers), cellular renewal, ketosis, weight loss, glycemic control, blood pressure, satiety, or treatment of gastroesophageal reflux disease (GERD). Another aspect is a method of treating or preventing an inflammatory condition, such as rheumatoid arthritis and/or osteoarthritis, by administering an autophagy inducer before, during and/or after an intermittent fasting (IF) diet. An example of an autophagy inducer suitable for the compositions and methods is thymol, optionally in combination with medium chain triglycerides (MCTs).

### TECHNICAL FIELD

The present disclosure generally relates to administering an autophagy inducer before, during and/or after an intermittent fasting (IF) diet, preferably a time-restricted feeding (TRF) regimen or an alternate day fasting (ADF) regimen. For example, enhanced intermittent fasting provided by the compositions and methods disclosed herein can comprise improvement of at least one of longevity, cardiometabolic health, body composition (e.g., fat mass reduction), cellular ageing (e.g., reduction in inflammation markers), cellular renewal, ketosis, weight loss, glycemic control, blood pressure, satiety, or treatment of gastroesophageal reflux disease (GERD).

### BACKGROUND

Intermittent fasting (IF) is a generic term to describe dietary patterns that include a period where little or no energy intake is consumed, alternating with a feeding-sometimes referred to as feasting-period.

One of the most popular styles of IF is the time-restricted feeding (TRF) regimen in which consumption of food is only allowed during a specified period of time per day, e.g., people on a 16:8 regimen fast for 16 hours and eat for 8 hours each day. Another popular style of IF is alternate day fasting (ADF) in which 24-hour fasting periods alternate with 24-hours of ad libitum intake. There are three common versions of ADF: (1) strict alternate day fasting in which people alternate one day of eating with one day of fasting; no calories are consumed on the fasting day, but non-caloric beverages (e.g., water, infusions, tea, coffee) may be consumed; (2) modified alternate day fasting in which people alternate one day of eating with one day of a modified fast, consuming foods and beverages with a limited number of calories (e.g., ≤500 kcal) on the fasting day; and (3) a 5:2 regimen in which people fast two out of every seven days, including modifications in which a limited number of calories can be consumed two out of seven days.

Generally used as a weight loss regimen, IF has also been purported to have other health benefits including glycemic control, cardiovascular health, and treatment of inflammatory conditions such as rheumatoid arthritis and osteoarthritis, based on animal and human research (De Cabo, R & Mattson, M. P. (2019). Effects of Intermittent Fasting on Health, Aging, and Disease. NEJM 381 (26) 2541-2551). Efficacy of IF on health parameters varies widely in the literature, most likely due to the variability in study subjects and study designs.

### SUMMARY

Fasting has been shown to trigger autophagy, which is a mechanism at the crossroad of intermittent fasting benefits such as longevity (Longo, V. D. & Panda, S. (2016) Fasting, circadian rhythms, and time-restricted feeding in healthy lifespan. Cell Metab. 23, 1048-1059). Indeed, autophagy is a cellular recycling process implicated in cellular renewal.

Existing nutritional solutions target ketosis with MCTs positioned for intermittent fasting. However, to the best knowledge of the present inventor, there are no solutions targeting autophagy to enhance intermittent fasting benefits. For example, known compositions for intermittent fasting can further include weight loss agent such as MCTs, but the end benefits are typically related to only weight loss. However, the present disclosure provides the combination of an autophagy ingredient inducer and an intermittent fasting regimen to enhance their related benefits, such as effects on longevity and cellular renewal.

Accordingly, compositions and methods disclosed herein can use an autophagy inducer for induction of autophagy in an individual before, during and/or after intermittent fasting (IF). Preferably, a formulation comprising an autophagy inducer is administered to the individual in an amount effective to induce autophagy, for example in muscle. The formulation can concomitantly promote protein synthesis and removal of damaged cellular materials.

In one or more embodiments, the autophagy inducer is selected from the group consisting of thymol, carvacrol, cannabidiolic acid (CBDA), spermidine, urolithin (e.g., Urolithin A, B or D), rapamycin, valproic acid, polyphenols (e.g., resveratrol, curcumin), Torin-1 (1-[4-(4-propanoylpiperazin-1-yl)-3-(trifluoromethyl)phenyl]-9-quinolin-3-ylbenzo[h][1,6]naphthyridin-2-one), caffeine, metformin, 5′ AMP-activated protein kinase (AMPK) activators, L-type calcium channel inhibitors, ketones (e.g., beta-hydroxybutyrate, ketone salts, or ketone ester derivatives), and mixtures thereof.

Non-limiting examples of suitable autophagy inducers are spermidine, palmitic acid, 5-aminoimidazole-4-carboxamide riboside (AICAR), verapamil, nifedipine, diltiazem, piperazine phenothiazine derivatives (e.g., trifluoperazine), ketones (e.g., beta-hydroxybutyrate, ketone salts, or ketone ester derivatives) and mixtures thereof. Non-limiting examples of suitable forms of spermidine include spermidine trihydrochloride, spermidine phosphate hexahydrate, spermidine phosphate hexahydrate, and L-arginyl-3, 4-spermidine.

Additionally or alternatively, the autophagy inducer can comprise one or more autophagy-inducing amino acids, for example Glycine, Cysteine, Proline, Glutamate (0.1-100 mg), Valine, Tyrosine or their precursors, such as Serine (as a precursor to Glycine), N-Acetyl Cysteine (0.1 to 100 mg), Methionine (as a precursor to Cysteine), and mixtures thereof. In addition to the one or more autophagy-inducing amino acids, optionally the composition can further comprise one or more anabolic amino acids such as Leucine, Isoleucine, Arginine, Glutamine or Citrulline. The composition can comprise the one or more anabolic amino acids in an amount effective to activate mTOR in the individual. In some embodiments, the composition comprises the one or more autophagy-inducing amino acids or their precursors in an amount effective for the composition to be at least neutral regarding autophagy and preferably positive regarding autophagy, despite any negative effect on autophagy from the one or more anabolic amino acids.

In some embodiments, an autophagy inducer, such as thymol, and optionally in combination with MCTs, promotes satiety; as supported by “Medium chain triglycerides and conjugated linoleic acids in beverage form increase satiety and reduce food intake in humans” by Coleman, H., P. Quinn, and M. E. Clegg, Nutrition Research 36(6):526-33 (2016) and “Substrate oxidation and control of food intake in men after a fat-substitute meal compared with meals supplemented with an isoenergetic load of carbohydrate, long-chain triacylglycerols, or medium-chain triacylglycerols” by Wymelbeke, V. V., J. Louis-Sylvestre, and M. Fantino, The American Journal of Clinical Nutrition 74(5):620-30 (2001).

Additional features and advantages are described herein and will be apparent from the following Figures and Detailed Description.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

As used in this disclosure and the appended claims, the singular forms “a,” “an” and “the” include plural referents unless the context clearly dictates otherwise. Thus, for example, reference to “an autophagy inducer” or “the autophagy inducer” encompass both an embodiment having a single autophagy inducer and an embodiment having two or more autophagy inducers.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions disclosed herein may lack any element that is not specifically disclosed herein. Thus, a disclosure of an embodiment using the term “comprising” includes a disclosure of embodiments “consisting essentially of” and “consisting of” the components identified.

The terms “at least one of” and “and/or” used in the respective context of “at least one of X or Y” and “X and/or Y” should be interpreted as “X,” or “Y,” or “X and Y.” For example, “at least one of a vitamin or mineral” and “vitamin and/or mineral” should be interpreted as “vitamin without mineral,” or “mineral without vitamin,” or “both vitamin and mineral.”

Where used herein, the terms “example” and “such as,” particularly when followed by a listing of terms, are merely exemplary and illustrative and should not be deemed to be exclusive or comprehensive. As used herein, a condition “associated with” or “linked with” another condition means the conditions occur concurrently, preferably means that the conditions are caused by the same underlying condition, and most preferably means that one of the identified conditions is caused by the other identified condition.

Examples of types of Time Restricted Feeding (TRF) include the following:
(1) 12:12—fasting for 12 hours and ad libitum eating for 12 hours,
(2) 16:8—fasting for 16 hours and ad libitum eating for 8 hours, and
(3) 20:4—fasting for 20 hours and ad libitum eating for 4 hours.

### Types of Alternate Day Fasting (ADF) Include the Following:

(1) Strict Alternate Day Fasting where people alternate one day of eating with one day of fasting. In this regimen, no calories are consumed on the fasting day, but non-caloric beverages (e.g., water, infusions, tea, coffee) may be consumed.

(2) Modified Alternate Day Fasting where people alternate one day of eating with one day of a modified fast, consuming foods and beverages with a limited number of calories (e.g. ≤500 kcal) on the fasting day.

(3) 5:2 regimen where people fast two out of every seven days. The 5:2 regimen also includes modifications where on two out of seven days, a limited number of calories can be consumed.

The term “composition” can mean a food, beverage, dietary supplement, complete nutrition or oral nutritional supplement (ONS) or medical food composition, or mixture thereof. Within the context of the present disclosure, “nutrients” are substances needed for health, growth, development and functioning of an organism, including: macronutrients (e.g., protein, carbohydrates, fats) and their components (e.g., amino acids, sugars, starches, fatty acids, etc.); micronutrients (e.g., vitamins, minerals); other food components (fiber, cholesterol, bioactive phytochemicals, alcohol, etc.); and water contained in foods and beverages.

Within the context of the present disclosure, the terms “food,” “food product” and “food composition” mean a product or composition that is intended for ingestion by an individual such as a human and provides nutritional support to an organism, including those that provide energy, nutrients, and water. The compositions of the present disclosure, including the many embodiments described herein, can comprise, consist of, or consist essentially of one or more autophagy inducers, as well as any additional or optional ingredients or components safe for human consumption and otherwise useful in a diet.

The term “food for special medical purpose (FSMP)” refers to formula foods specially processed and prepared in order to meet special needs for nutrient or diet of those suffering from food intake restriction, disorder of digestive absorption, disorder of metabolic or certain diseases. Such foods shall be used alone or together with other foods under the guidance of a doctor or clinical nutritionist. FSMP is special dietary food, not medicine, but not ordinarily eaten by normal people. It is specially developed by clinicians and nutritionists based on scientific facts after extensive medical research.

The term “oral nutritional supplement (ONS)” refers to sterile liquids, semi-solids or powders, which provide macro- and micro-nutrients. They are widely used within the acute and community health settings for individuals who are unable to meet their nutritional requirements through oral diet alone.

Within the context of the present disclosure, the term “beverage,” “beverage product” and “beverage composition” mean a potable liquid product or composition for ingestion by an individual such as a human and provides water and may also include one or more nutrients and other ingredients safe for human consumption to the individual.

Within the context of the present disclosure, “dietary supplements” are products taken by mouth that contain one or more dietary ingredient, such as vitamins, minerals, amino acids, fatty acids, fibers and/or herbs and other botanical ingredients used to supplement the diet. Dietary supplements come in many forms and may be available as tablets, capsules, powders, liquids, and formulated into specific foods, such as “energy” bars.

As used herein, “complete nutrition” contains sufficient types and levels of macronutrients (protein, fats and carbohydrates), micronutrients, and other food components to be sufficient to be a sole source of nutrition for the subject to which the composition is administered. Individuals can receive 100% of their nutritional requirements from such complete nutritional compositions.

A triglyceride (also known as a triacylglycerol or a triacylglyceride) is an ester that is derived from glycerol and three fatty acids. Fatty acids may be either unsaturated or saturated. Fatty acids which are not attached to other molecules are referred to as free fatty acids (FFA).

A medium-chain triglyceride (MCT) is a triglyceride in which all three fatty acid moieties are medium-chain fatty acid moieties. As defined herein, medium-chain fatty acids (MCFA) are fatty acids that have 6 to 14 carbon atoms, preferably 6 to 12 carbon atoms. Medium-chain fatty acids with 8 carbon atoms may be referred to herein as “C8 fatty acids” or “C8.” Medium-chain fatty acids with 10 carbon atoms may be referred to herein as “C10 fatty acids” or “C10.”

The term “fatty acid moiety” refers to the part of the MCT that originates from a fatty acid in an esterification reaction with glycerol. In a non-limiting example, an esterification reaction between glycerol and only octanoic acid would result in a MCT with octanoic acid moieties. In another non-limiting example, an esterification reaction between glycerol and only decanoic acid would result in a MCT with decanoic acid moieties.

“Prevention” includes reduction of risk, incidence and/or severity of a condition or disorder. The terms “treatment” and “treat” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms “treatment” and “treat” do not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a patient, a caregiver, a doctor, a nurse, or another healthcare professional.

As used herein, a prophylactically or therapeutically “effective amount” is an amount that prevents a deficiency, treats a disease or medical condition in an individual, or, more generally, reduces symptoms, manages progression of the disease, or provides a nutritional, physiological, or medical benefit to the individual. The relative terms “improved,” “increased,” “enhanced” and the like refer to the effects for intermittent fasting, from administration of the composition comprising an autophagy inducer, relative to a composition without the autophagy induce or with less of the autophagy inducer, but otherwise identical. For example, enhanced intermittent fasting provided by the compositions and methods disclosed herein can comprise improvement of at least one of longevity, cardiometabolic health, body composition, cellular ageing, cellular renewal, ketosis, weight loss, glycemic control, blood pressure, satiety, or treatment of gastroesophageal reflux disease (GERD).

A “subject” or “individual” is a mammal, preferably a human. It can also be an an animal. While the terms “subject” and “individual” are often used herein to refer to a human, the present disclosure is not so limited.

“Animal” includes, but is not limited to, mammals, which includes but is not limited to rodents, aquatic mammals, domestic animals such as dogs and cats, farm animals such as sheep, pigs, cows and horses, and humans. Where “animal,” “mammal” or a plural thereof is used, these terms also apply to any animal that is capable of the effect exhibited or intended to be exhibited by the context of the passage, e.g., an animal capable of autophagy.

The term “unit dosage form,” as used herein, refers to physically discrete units suitable as unitary dosages for human and animal subjects, each unit containing a predetermined quantity of the composition disclosed herein in an amount sufficient to produce the desired effect, in association with a pharmaceutically acceptable diluent, carrier or vehicle. The specifications for the unit dosage form depend on the particular compounds employed, the effect to be achieved, and the pharmacodynamics associated with each compound in the host.

### Embodiments

An aspect of the present disclosure is a method of enhancing an intermittent fasting (IF) diet in an individual before, during and/or after the IF diet, for example by improvement of at least one of longevity, cardiometabolic health, body composition, cellular ageing, cellular renewal, ketosis, weight loss, glycemic control, blood pressure, satiety, or treatment of gastroesophageal reflux disease (GERD). The method comprises administering an autophagy inducer (e.g., an effective amount of an autophagy inducer) to an individual (e.g., an individual in need thereof). The autophagy inducer can be administered in a composition which can be administered parenterally, enterally, or intravenously.

Another aspect of the present disclosure is a method of treating, preventing, and/or reducing at least one of a risk, an incidence of, or a severity of an inflammatory condition, such as rheumatoid arthritis and osteoarthritis, in an individual before, during and/or after an intermittent fasting (IF) diet. The method comprises administering an autophagy inducer (e.g., an effective amount of an autophagy inducer) to an individual (e.g., an individual in need thereof). The autophagy inducer can be administered in a composition which can be administered parenterally, enterally, or intravenously.

Preferably the autophagy inducer is orally administered to the individual in a beverage or as a food composition. Non-limiting examples of suitable compositions for the include food compositions, dietary supplements, dietary supplements (e.g., liquid ONS), complete nutritional compositions, beverages, pharmaceuticals, oral nutritional supplement, meal replacement, medical food, nutraceuticals, food for special medical purpose (FSMP), powdered nutritional products to be reconstituted in water or milk before consumption, food additives, medicaments, drinks, petfood, and combinations thereof.

In an embodiment, the unit dosage form is a predetermined amount of the beverage or the food composition (e.g., a predetermined amount of the beverage/food composition that comprises an effective amount of autophagy inducer).

Yet another embodiment is a method of making a composition for administration before, during and/or after an intermittent fasting (IF) diet, the method comprising adding an autophagy inducer to at least one other component. In some embodiments, the composition is formulated for oral administration, and the at least one other component is edible.

In some embodiments, the composition can be a ready to drink (RTD) beverage in a container, and the unit dosage form is a predetermined amount of the RTD beverage sealed in the container, which is opened for the oral administration. For example, the predetermined amount of the RTD beverage can comprise an effective amount of an autophagy inducer. An RTD beverage is a liquid that can be orally consumed without addition of any further ingredients.

In other embodiments, the method comprises forming the beverage by reconstituting a unit dosage form of a powder, which comprises the autophagy inducer, in a diluent such as water or milk to thereby form the beverage subsequently orally administered to the individual (e.g., within about ten minutes after reconstitution, within about five minutes after reconstitution, or within about one minute after reconstitution). The unit dosage form of the powder can be sealed in a sachet or other package, which can be opened for the reconstitution and subsequent oral administration.

The unit dosage form of the supplement can contain excipients, emulsifiers, stabilizers and mixtures thereof.

In one or more embodiments, the autophagy inducer is selected from the group consisting of thymol, carvacrol, cannabidiolic acid (CBDA), spermidine, urolithin (e.g., Urolithin A, B or D), rapamycin, valproic acid, polyphenols (e.g., resveratrol, curcumin), Torin-1 (1-[4-(4-propanoylpiperazin-1-yl)-3-(trifluoromethyl)phenyl]-9-quinolin-3-ylbenzo[h][1,6]naphthyridin-2-one), caffeine, metformin, 5′ AMP-activated protein kinase (AMPK) activators, L-type calcium channel inhibitors, ketones (e.g., beta-hydroxybutyrate, ketone salts, or ketone ester derivatives), and mixtures thereof.

In some embodiments, about 1 mg to about 1 g, preferably about 10 mg to 500 mg, more preferably about 20 mg to 200 mg thymol and/or carvacrol, per daily dose in one or more portions, is administered to the individual. In another embodiment, about 120 mg of thymol and/or carvacrol, per daily dose in one or more portions, is administered to the individual. Thymol (10-64%) is one of the major constituent of essential oils of thyme (Thymus vulgaris L., Lamiaceae). Carvacrol is present in the essential oil of Origanum vulgare (oregano), oil of thyme, oil obtained from pepperwort, and wild bergamot. The essential oil of thyme subspecies contains between 5% and 75% of carvacrol, while Satureja (savory) subspecies have a content between 1% and 45%. Origanum majorana (marjoram) and Dittany of Crete are rich in carvacrol, 50% and 60-80% respectively. Therefore, some embodiments of the composition comprise such plant and/or enriched plant extracts, essential oils or fractions that provide at least a portion of thymol and/carvacrol in the composition, in particular from thyme and oregano.

Further non-limiting examples of suitable autophagy inducers are urolithin, spermidine, palmitic acid, 5-aminoimidazole-4-carboxamide riboside (AICAR), verapamil, nifedipine, diltiazem, piperazine phenothiazine derivatives (e.g., trifluoperazine), ketones (e.g., beta-hydroxybutyrate, ketone salts, or ketone ester derivatives) and mixtures thereof. Non-limiting examples of suitable forms of spermidine include spermidine trihydrochloride, spermidine phosphate hexahydrate, spermidine phosphate hexahydrate, and L-arginyl-3, 4-spermidine.

The method can comprise administering daily the autophagy inducer (e.g., spermidine) in the weight range of 0.05 mg-1 g per kg body weight, preferably 1 mg-200 mg per kg body weight, more preferably 5 mg-150 mg per kg body weight, even more preferably 10 mg-120 mg per kg body weight, or most preferably 40 mg-80 mg per kg body weight.

In some embodiment, urolithin can be administered in an amount of about 0.2-150 milligram (mg) of urolithin per kilogram (kg) of body weight of the subject. Preferably, the urolithin is administered in a dose equal or equivalent to 2-120 mg of urolithin per kg body weight of the subject, more preferably 4-90 mg of urolithin per kg body weight of the subject, most preferably 8-30 mg of urolithin per kg body weight of the subject.

Additionally or alternatively, the autophagy inducer can comprise one or more autophagy-inducing amino acids, for example Glycine, Cysteine, Proline, Glutamate, Valine, Tyrosine or their precursors, such as Serine (as a precursor to Glycine), N-Acetyl Cysteine, Methionine (as a precursor to Cysteine), and mixtures thereof. In one embodiment, the glycine can be administered in an amount of about 0.1-100 milligram (mg) of glycine or functional derivative thereof per kilogram (kg) of body weight of the subject. A daily dose of the composition can include one or more of: 0.1-100 mg/kg body weight (bw) Glycine; 5-340 mg/kg bw Valine; 20-126 mg/kg bw Tyrosine; 3-43 mg/kg bw Methionine. The composition comprises the one or more autophagy-inducing amino acids or their precursors in an amount effective for the composition to be at least neutral regarding autophagy and preferably positive regarding autophagy.

In addition to the one or more autophagy-inducing amino acids, optionally the composition can further comprise one or more anabolic amino acids such as Leucine, Isoleucine, Arginine, Glutamine or Citrulline. A daily dose of the composition can include one or more of 0.175-142.85 mg/kg bw Leucine, preferably 0.35-71.425 mg/kg bw Leucine; 0.175-71.425 mg/kg bw Isoleucine; 20-300 mg/kg bw Arginine, preferably 50-200 mg/kg bw Arginine and/or 20-300 mg/kg bw Citrulline, preferably 100-200 mg/kg bw Citrulline. The daily dose of the one or more anabolic amino acids can be provided by one or more servings of the composition per day.

The composition can comprise the one or more anabolic amino acids in an amount effective to activate mTOR in the individual. In some embodiments, the composition comprises the one or more autophagy-inducing amino acids or their precursors in an amount effective for the composition to be at least neutral regarding autophagy and preferably positive regarding autophagy, despite any negative effect on autophagy from the one or more anabolic amino acids.

In some embodiments, the method comprises administering a composition disclosed by any of U.S. patent application Ser. No. 16/954,694 entitled “Compositions and Methods Using a Combination of Autophagy Inducer and High Protein for Induction of Autophagy,” WO2020/245299 entitled “Compositions and Methods Using One or More Autophagy-Inducing Amino Acids to Potentiate Musculoskeletal Effect of One or More Anabolic Amino Acids” or WO2020/254663 entitled “Compositions and Methods Using Thymol and/or Carvacrol For Induction of Autophagy,” each of which are incorporated by reference in their entireties.

In some embodiments, the composition is administered less one month before performing the IF regimen, for example less than one week before performing the IF regimen. Additionally or alternatively, the composition is administered at least the majority of the days during the performing of the IF regimen, for example daily during the performing of the IF regimen. Additionally or alternatively, the composition is administered for at least one week after performing the IF regimen, for example for at least one month after performing the IF regimen.

In particular embodiments, the method augments the plasma spermidine level in a subject before, during and/or after an IF regimen, for example to a level in the range of 50 to 6000 nmol/L plasma, preferably 100 to 6000 nmol/L plasma. The method can comprise administering a daily dose of the autophagy inducer (e.g., spermidine) in the weight range of 0.05 mg-1 g per kg body weight, preferably 1 mg-200 mg per kg body weight, more preferably 5 mg-150 mg per kg body weight, even more preferably 10 mg-120 mg per kg body weight, or most preferably 40 mg-80 mg per kg body weight.

Typically between 50 μg to 10 g of the autophagy inducer, preferably a spermidine compound, per daily dose in one or more portions is administered to a subject before, during and/or after an IF regimen.

Wheat germ is rich in spermidine. Therefore, some embodiments of the composition comprise wheat germ and/or enriched wheat germ extracts that provide at least a portion of the autophagy inducer in the composition.

Whey protein is rich in BCAAs such as Leucine and Isoleucine. Therefore, some embodiments of the composition comprise whey protein that provides at least a portion of the anabolic amino acids in the composition. Moreover, hydrolyzed collagen is a rich source of glycine and proline, and thus some embodiments of the composition comprise hydrolyzed collagen that provides at least a portion of the autophagy-inducing amino acids in the composition.

In a particular non-limiting embodiment, the composition comprises an autophagy inducer (preferably at least one of thymol oil or oregano oil), MCTs (preferably MCT oil), and optionally at least one additional component selected from the group consisting of protein (e.g., or more of collagen, pea protein), a gum (e.g., xanthan), one or more vitamins (e.g., at least one of Vitamin B12, Vitamin B1 or Vitamin D), one or more minerals (e.g., at least one of iron, zinc, or magnesium), a natural flavor, a natural color, and mixtures thereof.

The term “protein” as used herein includes free form amino acids, molecules between 2 and 20 amino acids (referenced herein as “peptides”), and also includes longer chains of amino acids as well. Small peptides, i.e., chains of 2 to 10 amino acids, are suitable for the composition alone or in combination with other proteins. The “free form” of an amino acid is the monomeric form of the amino acid. Suitable amino acids include both natural and non-natural amino acids. The composition can comprise a mixture of one or more types of protein, for example one or more (i) peptides, (ii) longer chains of amino acids, or (iii) free form amino acids; and the mixture is preferably formulated to achieve a desired amino acid profile/content.

At least a portion of the protein can be from animal or plant origin, for example dairy protein such as one or more of milk protein, e.g., milk protein concentrate or milk protein isolate; caseinates or casein, e.g., micellar casein concentrate or micellar casein isolate; or whey protein, e.g., whey protein concentrate or whey protein isolate. Additionally or alternatively, at least a portion of the protein can be plant protein such as one or more of soy protein or pea protein.

Mixtures of these proteins are also suitable, for example mixtures in which casein is the majority of the protein but not the entirety, mixtures in which whey protein is the majority of the protein but not the entirety, mixtures in which pea protein is the majority of the protein but not the entirety, and mixtures in which soy protein is the majority of the protein but not the entirety. In an embodiment, at least 10 wt. % of the protein is whey protein, preferably at least 20 wt. %, and more preferably at least 30 wt. %. In an embodiment, at least 10 wt. % of the protein is casein, preferably at least 20 wt. %, and more preferably at least 30 wt. %. In an embodiment, at least 10 wt. % of the protein is plant protein, preferably at least 20 wt. %, more preferably at least 30 wt. %.

Whey protein may be any whey protein, for example selected from the group consisting of whey protein concentrates, whey protein isolates, whey protein micelles, whey protein hydrolysates, acid whey, sweet whey, modified sweet whey (sweet whey from which the caseino-glycomacropeptide has been removed), a fraction of whey protein, and any combination thereof.

Casein may be obtained from any mammal but is preferably obtained from cow milk and preferably as micellar casein.

The protein may be unhydrolyzed, partially hydrolyzed (i.e., peptides of molecular weight 3 kDa to 10 kDa with an average molecular weight less than 5 kDa) or extensively hydrolyzed (i.e., peptides of which 90% have a molecular weight less than 3 kDa), for example in a range of 5% to 95% hydrolyzed. In some embodiments, the peptide profile of hydrolyzed protein can be within a range of distinct molecular weights. For example, the majority of peptides (>50 molar percent or >50 wt. %) can have a molecular weight within 1-5 kDa, or 5-10 kDa, or 10-20 kDa.

In an embodiment, the composition includes a source of carbohydrates. Any suitable carbohydrate may be used in the composition including, but not limited to, starch (e.g., modified starch, amylose starch, tapioca starch, corn starch), sucrose, lactose, glucose, fructose, corn syrup solids, maltodextrin, xylitol, sorbitol or combinations thereof.

The source of carbohydrates is preferably not greater than 50 energy % of the composition, more preferably not greater than 36 energy % of the composition, and most preferably not greater than 30 energy % of the composition.

In an embodiment, the composition includes a source of fat. The source of fat may include any suitable fat or fat mixture. Non-limiting examples of suitable fat sources include vegetable fat, such as olive oil, corn oil, sunflower oil, high-oleic sunflower, rapeseed oil, canola oil, hazelnut oil, soy oil, palm oil, coconut oil, blackcurrant seed oil, borage oil, lecithins, and the like, animal fats such as milk fat; or combinations thereof. The composition comprising an autophagy inducer (e.g., spermidine) can be administered to an individual before, during and/or after intermittent fasting (IF), in a therapeutically effective dose. The therapeutically effective dose can be determined by the person skilled in the art and will depend on a number of factors known to those of skill in the art, such as the severity of the condition and the weight and general state of the individual.

The composition is preferably administered to the individual at least two days per week, more preferably at least three days per week, most preferably all seven days of the week; for at least one week, at least one month, at least two months, at least three months, at least six months, or even longer. In some embodiments, the composition is administered to the individual consecutively for a number of days, for example at least until a therapeutic effect is achieved. In an embodiment, the composition can be administered to the individual daily for at least 30, 60 or 90 consecutive days.

The above examples of administration do not require continuous daily administration with no interruptions. Instead, there may be some short breaks in the administration, such as a break of two to four days during the period of administration. The ideal duration of the administration of the composition can be determined by those of skill in the art.

In a preferred embodiment, the composition is administered to the individual orally or enterally (e.g. tube feeding). For example, the composition can be administered to the individual as a beverage, a capsule, a tablet, a powder or a suspension.

The composition can be any kind of composition that is suitable for human and/or animal consumption. For example, the composition may be selected from the group consisting of food compositions, dietary supplements, nutritional compositions, nutraceuticals, powdered nutritional products to be reconstituted in water or milk before consumption, food additives, medicaments, beverages and drinks. In an embodiment, the composition is an oral nutritional supplement (ONS), a complete nutritional formula, a pharmaceutical, a medical or a food product. In a preferred embodiment, the composition is administered to the individual as a beverage. The composition may be stored in a sachet as a powder and then suspended in a liquid such as water for use.

In particular non-limiting examples, the preferred formats of the composition are beverages (e.g., comprising water and/or another liquid), ready-to-drink beverages which do not require addition of any other component before consumption (e.g., comprising water and/or coffee); oral nutritional supplement (ONS); or a coffee creamer.

In some instances where oral or enteral administration is not possible or not advised, the composition may also be administered parenterally. Preferred parenteral administration is intravenous administration. A particular form of parenteral administration is delivery by intravenous administration of nutrition. Parenteral nutrition is “total parenteral nutrition” when no food is given by other routes. “Parenteral nutrition” is preferably a isotonic or hypertonic aqueous solution (or solid compositions to be dissolved, or liquid concentrates to be diluted to obtain an isotonic or hypertonic solution) comprising a saccharide such as glucose and further comprising one or more of lipids, amino acids, and vitamins.

In some embodiments, the composition is administered to the individual in a single dosage form, i.e., all compounds are present in one product to be given to an individual in combination with a meal. In other embodiments, the composition is co-administered in separate dosage forms, for example at least one component separately from one or more of the other components of the composition.

In view of the disclosures herein, an embodiment provided by the present disclosure is a composition comprising an autophagy inducer and formulated for administration to an individual before, during and/or after an intermittent fasting (IF) regimen, preferably a time-restricted feeding (TRF) regimen or an alternate day fasting (ADF) regimen, wherein the composition preferably further comprises medium chain triglycerides (MCTs) and optionally at least one additional ingredient selected from the group consisting of protein, a gum, a vitamin, a mineral, a natural flavor, and a natural color.

Preferably, the autophagy inducer comprises at least one of thymol, preferably at least partially provided by thyme oil or oregano oil, in the composition, or carvacrol, preferably at least partially provided by oregano oil in the composition. In some embodiments, the composition comprises protein, preferably at least one of collagen, pea protein). In some embodiments, the composition comprises a gum, preferably xanthan. In some embodiments, the composition comprises a vitamin, preferably at least one of Vitamin B1, Vitamin B12, or Vitamin D. In some embodiments, the composition comprises a mineral, preferably at least one of iron, zinc or magnesium.

In some embodiments, the autophagy inducer is selected from the group consisting of thymol, carvacrol, cannabidiolic acid (CBDA), spermidine, urolithin, rapamycin, valproic acid, a polyphenol, caffeine, metformin, a ketone, palmitic acid, 1-[4-(4-propanoylpiperazin-1-yl)-3-(trifluoromethyl)phenyl]-9-quinolin-3-ylbenzo[h][1,6]naphthyridin-2-one, a 5′ AMP-activated protein kinase (AMPK) activator, an L-type calcium channel inhibitor, 5-aminoimidazole-4-carboxamide riboside (AICAR), verapamil, nifedipine, diltiazem, piperazine phenothiazine derivatives, one or more autophagy-inducing amino acids, and mixtures thereof.

Preferably, the composition has a form selected from the group consisting of a beverage, an oral nutritional supplement (ONS), and a coffee creamer. The composition may also be a for animal consumption.

Compositions intended for a non-human animal include food compositions to supply the necessary dietary requirements for an animal, animal treats (e.g., biscuits), and/or dietary supplements. The compositions may be a dry composition (e.g., kibble), semi-moist composition, wet composition, or any mixture thereof. In one embodiment, the composition is a dietary supplement such as a gravy, drinking water, beverage, yogurt, powder, granule, paste, suspension, chew, morsel, treat, snack, pellet, pill, capsule, tablet, or any other suitable delivery form. In one embodiment, another suitable delivery form may include encapsulation of at least one of the active ingredients of the composition, by means of macro or microencapsulation. Microencapsulation encompasses e.g. microcapsules, microparticles, microspheres, and microemulsions. Macro and microencapsulation technologies, including chemical, physicochemical or mechanical methods, are well known in the art.

The dietary supplement may require admixing, or can be admixed with water or other diluent prior to administration to the animal.

Another embodiment is a unit dosage form of any of the compositions disclosed herein, the unit dosage form comprising an amount of the autophagy inducer that is effective to enhance an intermittent fasting (IF) diet for an individual to whom the unit dosage form is administered. Preferably, the unit dosage form of Claim 9, wherein the enhancement to the IF diet comprises improvement of at least one of longevity, cardiometabolic health, body composition, cellular ageing, cellular renewal, ketosis, weight loss, glycemic control, blood pressure, satiety, or treatment of gastroesophageal reflux disease (GERD).

Another embodiment is a method of enhancing an intermittent fasting (IF) diet, the method comprising administering any of the compositions disclosed herein to a subject before, during and/or after the IF diet. Preferably, the composition is administered daily to the subject for at least one week.

Another embodiment is a method of making a composition for administration before, during and/or after an intermittent fasting (IF) diet, the method comprising adding an autophagy inducer to at least one other component selected from the group consisting of medium chain triglycerides (MCTs), protein, a gum, a vitamin, a mineral, a natural flavor, and a natural color.

### Example

The following non-limited example is representative of one or more embodiments disclosed herein.

### Worm Survival Assay on Solid Agar Plate

C. elegans strains were cultured at 20° C. on nematode growth media agar plates seeded with E. coli strain OP50. Thymol was dissolved in DMSO and added at the indicated concentrations just before pouring the plates. Worms were exposed to compounds during the full life from eggs until death. To ensure a permanent exposure to the compound, plates were changed twice a week. The control population was treated with the corresponding concentration of DMSO at 1%. For lifespan measurements, parental F0 L4 worms were grown to reach adulthood and lay eggs on the treatment plates. The derived F1 worms were therefore exposed to compounds during the full life from eggs until death. Adults were daily scored manually as dead or alive. A worm was considered dead if it was not moving spontaneously and it was not responding to a touch on the head. The control population was treated with the corresponding concentration of DMSO at 1%.

### Worm Survival Assay in Microfluidics

A worm suspension is first injected into a microfluidic device. The geometry of the device is optimized for retaining inside the worm culture chamber only adult worms by simply selecting the correct flow rate for the sample injection. Upon isolation of a defined worm population inside the chamber, worms are cultured and treated on-chip with thymol at controlled temperature of 20° C. Worm culture is maintained in the chamber by the perfusion of E. coli. The microfluidic chip is integrated onto an inverted microscope integrated with a camera that continuously records videos (every 10 hours). Images are longitudinally analyzed by different parameters that include readouts of body bends frequency, velocity, curvature and amplitude of the movement at the head, tail and middle of the body, which automatically provide information about worms health span and vitality.

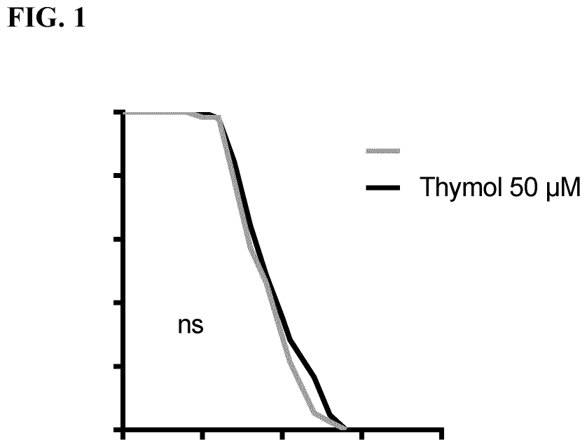

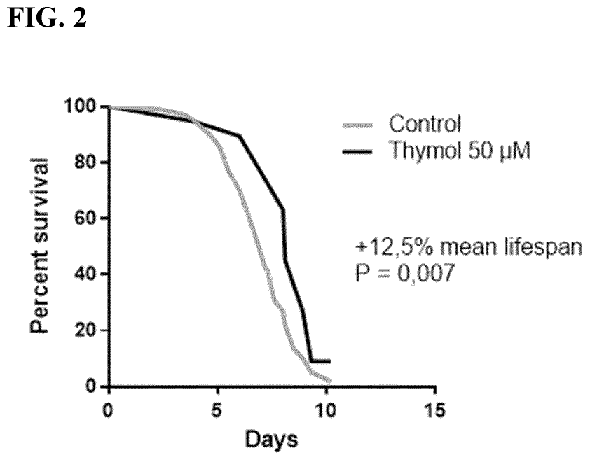

FIG. 1 shows that treatment of worms with thymol at a concentration of 50 M on solid agar plates has no significant effects on lifespan (n=60 per group). FIG. 2 shows that thymol increases lifespan at a concentration of 50 μM when the worms are treated in microfluidics. P-values represent comparison with vehicle calculated using log rank test. Thymol alone (control) does not improve lifespan statistically, and the effect on lifespan is statistically significant when the worms are in conditions mimicking the IF.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method of enhancing an intermittent fasting (IF) diet, the method comprising administering a composition to a subject before, during and/or after the IF diet, the composition comprising an autophagy inducer.

2. The method of claim 1, wherein the autophagy inducer comprises at least one of thymol.

3. The method of claim 1, wherein the composition further comprises medium chain triglycerides (MCTs)

4. The method of claim 1, wherein the composition comprises at least one additional ingredient selected from the group consisting of protein, a gum, a vitamin, a mineral, a natural flavor, and a natural color.

5. The method of claim 1, wherein the composition comprises a vitamin.

6. The method of claim 1, wherein the composition comprises a mineral.

7. The method of claim 1, wherein the autophagy inducer is selected from the group consisting of thymol, carvacrol, cannabidiolic acid (CBDA), spermidine, urolithin, rapamycin, valproic acid, a polyphenol, caffeine, metformin, 1-[4-(4-propanoylpiperazin-1-yl)-3-(trifluoromethyl)phenyl]-9-quinolin-3-ylbenzo[h][1,6]naphthyridin-2-one, a 5′ AMP-activated protein kinase (AMPK) activator, an L-type calcium channel inhibitor, a ketone, palmitic acid, 5-aminoimidazole-4-carboxamide riboside (AICAR), verapamil, nifedipine, diltiazem, piperazine phenothiazine derivatives, one or more autophagy-inducing amino acids, and mixtures thereof.

8. The method of claim 1, wherein the composition is in a form selected from the group consisting of a meal replacement, dietary supplements, complete nutritional compositions, beverages, pharmaceuticals, oral nutritional supplement, medical food, nutraceuticals, food for special medical purpose (FSMP), powdered nutritional products to be reconstituted in water or milk before consumption, food additives, medicaments, drinks, petfood, and combinations thereof.

9. The method of claim 8, wherein the dietary supplement is selected from the group consisting of a gravy, drinking water, beverage, yogurt, powder, granule, paste, suspension, chew, morsel, treat, snack, pellet, pill, capsule, and a tablet.

10. (canceled)

11. The method of claim 1, wherein the enhancement to the IF diet comprises improvement of at least one of longevity, cardiometabolic health, body composition, cellular ageing, cellular renewal, ketosis, weight loss, glycemic control, blood pressure, satiety, or treatment of gastroesophageal reflux disease (GERD).

12. (canceled)

13. A method of claim 1 including treating, and preventing a risk, an incidence, or a severity of an inflammatory condition.

14. The method of claim 1, wherein the inflammatory condition comprises at least one of rheumatoid arthritis or osteoarthritis.

15. The method of claim 1, wherein the composition is administered daily to the subject for at least one week.

16. A method of making a composition for administration before, during and/or after an intermittent fasting (IF) diet, the method comprising adding an autophagy inducer to at least one other component selected from the group consisting of medium chain triglycerides (MCTs), protein, a gum, a vitamin, a mineral, a natural flavor, and a natural color.
